SEQUENCE LISTING (1 sequence)

SEQ ID NO: 1 - protein, 756 aa, human
MSWSPSLTTQTCGAWEMKERLGTGGFGNVIRWHNQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

(19) United States
(12) Patent Application Publication
(10) Pub. No.: US 2013/0210875 A1
(43) Pub. Date: Aug. 15, 2013

(54) Novel binding site of IKK-beta

(71) Applicants: Liang LIU, Macau (CN); Ting LI, Macau (CN); Kam Wai WONG, Macau (CN); Zhihong JIANG, Macau (CN); Hua ZHOU, Macau (CN)

(72) Inventors: Liang LIU, Macau (CN); Ting LI, Macau (CN); Kam Wai WONG, Macau (CN); Zhihong JIANG, Macau (CN); Hua ZHOU, Macau (CN)

(73) Assignee: MACAU UNIVERSITY OF SCIENCE AND TECHNOLOGY, Macau (CN)

(21) Appl. No.: 13/370,313

(22) Filed: Feb. 10, 2012

(51) Int. Cl. A61K 31/4188; G01N 33/574; A61K 31/352; A61P 35/00; A61P 29/00; A61P 19/06; A61P 37/00; A61P 19/02; A61P 35/02; A61P 25/28; A61P 25/16; A61P 11/06; G01N 33/573; A61P 25/00

(52) U.S. Cl. 514/387; 435/7.8; 514/456

(57) ABSTRACT

Uses and applications derived from the discovery of a novel binding site of IKK-β, such as method of screening a therapeutic agent as drug candidate for treating cancer, inflammation, or other diseases/disorders, are provided.

### FIELD OF INVENTION

This invention relates to a novel binding site of IKK-β, and in particular uses and applications derived from the discovery of this novel binding site.

### BACKGROUND OF INVENTION

Diseases such as cancers and inflammations are deliberating and may be fatal. Thus, it is essential to develop methods for effectively screening therapeutic agents as drug candidates for treating these diseases. It is also important to develop reliable methods for screening and/or diagnosing patients having these disorders so that they can receive appropriate treatment as early as possible.

### SUMMARY OF INVENTION

In the light of the foregoing background, it is an object of the present invention to provide a new method for screening therapeutic agents as drug candidates for treating these diseases.

Accordingly, the present invention, in one aspect, is a method of screening a therapeutic agent as a drug candidate for treating cancer, inflammation, neurodegenerative disease, immunological disorder, or arthritic disorder comprising:

a) exposing said agent to an assay comprising IKK-β;

b) detecting whether said agent binds to cysteine-46 (Cys-46 or C46) residue of IKK-β;

c) detecting whether said agent inhibits kinase activity of IKK-β upon said binding in step (b); and

d) identifying a drug candidate that performs said binding action of step (b) and said inhibition action of step (c).

In an exemplary embodiment of the present invention, at least one binding site of IKK-β is mutated. In a further exemplary embodiment, said mutated binding site is selected from a group consisting of, phenylalanine residue, serine-177/181 residue, allosteric binding site of IKK-β, and cysteine residue except cysteine-46 residue.

In an even further exemplary embodiment, said mutated cysteine or phenylalanine residue is selected from a group consisting of cysteine-12 (Cys-12 or C12) residue, phenylalanine-26 (Phe-26 or F26, alternatively known as ATP binding site) residue, cysteine-59 (Cys-59 or C59) residue, cysteine-99 (Cys-99 or C99) residue, cysteine-114 (Cys-114 or C114) residue, cysteine-115 (Cys-115 or C115) residue, cysteine-179 (Cys-179 or C179) residue, cysteine-215 (Cys-215 or C215) residue, cysteine-299 (Cys-299 or C299) residue, cysteine-370 (Cys-370 or C370) residue, cysteine-412 (Cys-412 or C412) residue, cysteine-444 (Cys-444 or C444) residue, cysteine-464 (Cys-464 or C464) residue, cysteine-524 (Cys-524 or C524) residue, cysteine-618 (Cys-618 or C618) residue, cysteine-662/716 (Cys-662/716 or C662/716) residue, cysteine-751 (Cys-751 or C751) residue; said mutation is a point mutation from cysteine or phenylalanine to alanine.

In another embodiment, said cancer is selected from a group consisting of lung cancer, colon cancer, liver cancer, breast cancer, prostate cancer, cervical cancer, acute promyelocytic leukemia (APL), acute myeloid leukemia (AML), acute lymphocytic leukemia (ALL), chronic myelogenous leukemia (CML), non-Hodgkin's lymphoma, Hodgkin's disease, chronic lymphocytic leukemia (CLL), myelodysplastic syndrome, Adult T-cell leukemia (ATL), Burkitt's lymphoma, B-cell lymphoma, primary malignant lymphocytes, B-cell chronic lymphocytic leukemia (B-CLL), human THP-1 leukemia and multiple myeloma. In yet another embodiment, said inflammation is selected from a group consisting of ear edema, dermatitis, ear inflammation, and arthritis.

In another exemplary embodiment, said neurodegenerative disease is selected from a group consisting of Alzheimer's disease, Parkinson's disease, amyotrophic lateral sclerosis, ataxia telangiectasia, spinocerebellar atrophy, multiple sclerosis, and Huntington's chorea.

In yet another exemplary embodiment, said immunological disorder is selected from a group consisting of allergic rhinitis, allergic dermatitis, allergic contact dermatitis, allergic shock, asthma, papular urticaria, leucoderma, hypersensitivity vasculitis, hypersensitivity pneumonia, ulcerative colitis, glomerulonephritis, drug rashes, systemic lupus erythematosus, rheumatoid arthritis, scleroderma, multiple sclerosis, hyperthyroidism, idiopathic thrombocytopenic, autoimmune hemolytic anemia, allograft rejection, and hemolytic transfusion reaction.

In a further exemplary embodiment, aid arthritic disorder is selected from a group consisting of rheumatoid arthritis, ankylosing spondylitis, gout, periarthritis, osteoarthritis, Reiter syndrome, psoriatic arthritis, post-traumatic arthritis, and enteropathic arthritis.

According to another aspect of the present invention, a method for diagnosing cancer, inflammation, neurodegenerative disease, immunological disorder, or arthritic disorder in a patient is provided, comprising:

a) obtaining a sample from said patient;

b) contacting said sample with a compound that binds to cysteine-46 residue of IKK-β of said sample;

c) detecting binding of said compound to IKK-β in said sample;

d) detecting inhibition action on kinase activity of IKK-β by said compound upon said binding in step (c); and

e) diagnosing said patient as having a likelihood to develop cancer, inflammation, neurodegenerative disease, immunological disorder, or arthritic disorder if said compound cannot perform said binding action of step (c) and/or said inhibition action of step (d).

In an exemplary embodiment of the present invention, at least one binding site of IKK-β is mutated. In a further exemplary embodiment, said mutated binding site is selected from a group consisting of, phenylalanine residue, serine-177/181 residue, allosteric binding site, and cysteine residue except cysteine-46 residue.

In an even further exemplary embodiment, said mutated cysteine or phenylalanine residue is selected from a group consisting of cysteine-12 residue, phenylalanine-26 (Phe-26 or F26, alternatively known as ATP binding site) residue, cysteine-59 residue, cysteine-99 residue, cysteine-114 residue, cysteine-115 residue, cysteine-179 residue, cysteine-215 residue, cysteine-299 residue, cysteine-370 residue, cysteine-412 residue, cysteine-444 residue, cysteine-464 residue, cysteine-524 residue, cysteine-618 residue, cysteine-662/716 residue, and cysteine-751 residue; said mutation is a point mutation from cysteine or phenylalanine to alanine.

In another embodiment, said cancer is selected from a group consisting of lung cancer, colon cancer, and liver cancer, breast cancer, prostate cancer, cervical cancer, acute promyelocytic leukemia (APL), acute myeloid leukemia (AML), acute lymphocytic leukemia (ALL), chronic myelogenous leukemia (CML), non-Hodgkin's lymphoma, Hodgkin's disease, chronic lymphocytic leukemia (CLL), myelodysplastic syndrome, Adult T-cell leukemia (ATL), Burkitt's lymphoma, B-cell lymphoma, primary malignant lymphocytes, B-cell chronic lymphocytic leukemia (B-CLL), human THP-1 leukemia, and multiple myeloma. In yet another embodiment, said inflammation is selected from a group consisting of ear edema, dermatitis, ear inflammation, and arthritis.

In another exemplary embodiment, said neurodegenerative disease is selected from a group consisting of Alzheimer's disease, Parkinson's disease, amyotrophic lateral sclerosis, ataxia telangiectasia, spinocerebellar atrophy, multiple sclerosis, and Huntington's chorea.

In yet another exemplary embodiment, said immunological disorder is selected from a group consisting of allergic rhinitis, allergic dermatitis, allergic contact dermatitis, allergic shock, asthma, papular urticaria, leucoderma, hypersensitivity vasculitis, hypersensitivity pneumonia, ulcerative colitis, glomerulonephritis, drug rashes, systemic lupus erythematosus, rheumatoid arthritis, scleroderma, multiple sclerosis, hyperthyroidism, idiopathic thrombocytopenic, autoimmune hemolytic anemia, allograft rejection, and hemolytic transfusion reaction.

In a further exemplary embodiment, aid arthritic disorder is selected from a group consisting of rheumatoid arthritis, ankylosing spondylitis, gout, periarthritis, osteoarthritis, Reiter syndrome, psoriatic arthritis, post-traumatic arthritis, and enteropathic arthritis.

In a further aspect of the present invention, a method of screening a patient to have a likelihood to develop cancer, inflammation, neurodegenerative disease, immunological disorder, or arthritic disorder is provided, comprising:

a) obtaining a sample from said patient;

b) contacting said sample with a compound that binds to cysteine-46 residue of IKK-β of said sample;

c) detecting binding of said compound to IKK-β in said sample;

d) detecting inhibition action on kinase activity of IKK-β by said compound upon said binding in step (d); and

e) identifying said patient as having a likelihood to develop cancer, inflammation, neurodegenerative disease, immunological disorder, or arthritic disorder if said compound cannot perform said binding action of step (c) and/or said inhibition action of step (d).

In an exemplary embodiment of the present invention, at least one binding site of IKK-β is mutated. In a further exemplary embodiment, said mutated binding site is selected from a group consisting of, phenylalanine residue, serine-177/181 residue, allosteric binding site of IKK-β, and cysteine residue except cysteine-46 residue. In an even further exemplary embodiment, said mutated cysteine or phenylalanine residue is selected from a group consisting of cysteine-12 residue, phenylalanine-26 (Phe-26 or F26, alternatively known as ATP binding site) residue, cysteine-59 residue, cysteine-99 residue, cysteine-114 residue, cysteine-115 residue, cysteine-179 residue, cysteine-215 residue, cysteine-299 residue, cysteine-370 residue, cysteine-412 residue, cysteine-444 residue, cysteine-464 residue, cysteine-524 residue, cysteine-618, cysteine-662/716 residue, and cysteine-751 residue; said mutation is a point mutation from cysteine or phenylalanine to alanine.

In another embodiment, said cancer is selected from a group consisting of lung cancer, colon cancer, liver cancer, breast cancer, prostate cancer, cervical cancer, acute promyelocytic leukemia (APL), acute myeloid leukemia (AML), acute lymphocytic leukemia (ALL), chronic myelogenous leukemia (CML), non-Hodgkin's lymphoma, Hodgkin's disease, chronic lymphocytic leukemia (CLL), myelodysplastic syndrome, Adult T-cell leukemia (ATL), Burkitt's lymphoma, B-cell lymphoma, primary malignant lymphocytes, B-cell chronic lymphocytic leukemia (B-CLL), human THP-1 leukemia and, multiple myeloma. In yet another embodiment, said inflammation is selected from a group consisting of ear edema, dermatitis, ear inflammation, and arthritis.

In another exemplary embodiment, said neurodegenerative disease is selected from a group consisting of Alzheimer's disease, Parkinson's disease, amyotrophic lateral sclerosis, ataxia telangiectasia, spinocerebellar atrophy, multiple sclerosis, and Huntington's chorea.

In yet another exemplary embodiment, said immunological disorder is selected from a group consisting of allergic rhinitis, allergic dermatitis, allergic contact dermatitis, allergic shock, asthma, papular urticaria, leucoderma, hypersensitivity vasculitis, hypersensitivity pneumonia, ulcerative colitis, glomerulonephritis, drug rashes, systemic lupus erythematosus, rheumatoid arthritis, scleroderma, multiple sclerosis, hyperthyroidism, idiopathic thrombocytopenic, autoimmune hemolytic anemia, allograft rejection, and hemolytic transfusion reaction.

In a further exemplary embodiment, aid arthritic disorder is selected from a group consisting of rheumatoid arthritis, ankylosing spondylitis, gout, periarthritis, osteoarthritis, Reiter syndrome, psoriatic arthritis, post-traumatic arthritis, and enteropathic arthritis.

In yet a further aspect of the present invention, a method for treating cancer, inflammation, neurodegenerative disease, immunological disorder, or arthritic disorder is provided, comprising administering an effective amount of a therapeutic agent to a patient in need thereof, wherein said patient harbors gene mutations on at least one binding site of IKK-β; said mutated binding site is selected from a group consisting of phenylalanine residue, serine-177/181 residue, allosteric binding site of IKK-β, and cysteine residue except cysteine-46 residue.

In an exemplary embodiment, said mutated cysteine or phenylalanine residue is selected from a group consisting of cysteine-12 residue, phenylalanine-26 (Phe-26 or F26, alternatively known as ATP binding site) residue, cysteine-59 residue, cysteine-99 residue, cysteine-114 residue, cysteine-115 residue, cysteine-179 residue, cysteine-215 residue, cysteine-299 residue, cysteine-370 residue, cysteine-412 residue, cysteine-444 residue, cysteine-464 residue, cysteine-524 residue, cysteine 618 residue, cysteine-662/716 residue, and cysteine-751 residue; said mutation is a point mutation from cysteine or phenylalanine to alanine. In another embodiment, said therapeutic agent binds to cysteine-46 residue of IKK-β and inhibits the kinase activity of IKK-β upon said binding.

In another embodiment, said cancer is selected from a group consisting of lung cancer, colon cancer, liver cancer, breast cancer, prostate cancer, cervical cancer, acute promyelocytic leukemia (APL), acute myeloid leukemia (AML), acute lymphocytic leukemia (ALL), chronic myelogenous leukemia (CML), non-Hodgkin's lymphoma, Hodgkin's disease, chronic lymphocytic leukemia (CLL), myelodysplastic syndrome, Adult T-cell leukemia (ATL), Burkitt's lymphoma, B-cell lymphoma, primary malignant lymphocytes, B-cell chronic lymphocytic leukemia (B-CLL), human THP-1 leukemia and multiple myeloma. In yet another embodiment, said inflammation is selected from a group consisting of ear edema, dermatitis, ear inflammation, and arthritis.

In another exemplary embodiment, said neurodegenerative disease is selected from a group consisting of Alzheimer's disease, Parkinson's disease, amyotrophic lateral sclerosis, ataxia telangiectasia, spinocerebellar atrophy, multiple sclerosis, and Huntington's chorea.

In yet another exemplary embodiment, said immunological disorder is selected from a group consisting of allergic rhinitis, allergic dermatitis, allergic contact dermatitis, allergic shock, asthma, papular urticaria, leucoderma, hypersensitivity vasculitis, hypersensitivity pneumonia, ulcerative colitis, glomerulonephritis, drug rashes, systemic lupus erythematosus, rheumatoid arthritis, scleroderma, multiple sclerosis, hyperthyroidism, idiopathic thrombocytopenic, autoimmune hemolytic anemia, allograft rejection, and hemolytic transfusion reaction.

In a further exemplary embodiment, aid arthritic disorder is selected from a group consisting of rheumatoid arthritis, ankylosing spondylitis, gout, periarthritis, osteoarthritis, Reiter syndrome, psoriatic arthritis, post-traumatic arthritis, and enteropathic arthritis.

### BRIEF DESCRIPTION OF FIGURES

FIG. 1 shows the amino acid sequence of IKK-β protein as shown in SEQ ID NO: 1 in which the mutated residues are underlined.

FIG. 2A shows the synthesis of biotinylated DMY (DMY-biotin), whereas FIGS. 2B to 2D show the comparison of synthesized DMY-biotin and DMY on T cell proliferation, NF-κB activation, as well as IKK-β kinase activity according to one embodiment of the present invention.

FIG. 3 shows the study of the binding site of DMY and DMY-biotin according to one embodiment of the present invention.

FIG. 4 shows the study of a new drug binding site involved in IKK-β using IKK-β displacement binding assay according to one embodiment of the present invention.

FIG. 5 shows the study of DMY on its activity on drug resistant phenotype of IKK-β mutants with cysteine-179 mutation (C179A) or ATP-binding site mutation (F26A) according to one embodiment of the present invention.

FIG. 6 shows the study of DMY on its inhibition of the kinase activity of IKK-β mutants with cysteine-46 mutation (C46A), as well as form protein adduct with IKK-β mutants (C46A) according to one embodiment of the present invention.

FIG. 7 shows the study of DMY on its activity to suppress IKK-β mutants with various single cysteine mutations according to one embodiment of the present invention.

FIG. 8 shows the study of DMY on its ability to form protein adduct with IKK-β mutants with various single cysteine mutations according to one embodiment of the present invention.

FIG. 9 shows the study of DMY on its ability to suppress IKK-β-NF-κB signaling of both wild-type and IKK-β mutants with cysteine-46 mutation (C46A) in IKK-β−/− deficient MEFs according to one embodiment of the present invention.

FIGS. 10A to 10D show the study of DMY on its effect on ear edema induced by dinitrofluorobenzene according to one embodiment of the present invention.

FIGS. 11A to 11D show the study of DMY on its effect on arthritis model induced by collagen II according to one embodiment of the present invention.

### DETAILED DESCRIPTION OF THE PREFERRED EMBODIMENTS

As used herein and in the claims, “comprising” means including the following elements but not excluding others. When interpreting each statement in this specification that includes the term “comprising”, features other than that or those prefaced by the term may also be present. Related terms such as “comprise” and “comprises” are to be interpreted in the same manner.

The present invention provides a new technical platform for identifying the action mechanisms of existing IKK-β inhibitors and screening of new IKK-β inhibitors. In one exemplary embodiment, using the platform, dihydromyricetin (DMY) could directly suppress the kinase activity of IKK-β via novel drug binding site, cysteine-46 (Cys-46) residue, rather than via known binding sites on IKK-β such as ATP binding site, cysteine-179 (Cys-179) residue, serine-177/181 (Ser-177/181) residue, and allosteric binding site. In another exemplary embodiment, DMY could circumvent the drug resistance phenotype of IKK-β with mutations of Cys-179 residue or phenylalanine-26 (Phe-26 or F26, alternatively known as ATP binding site) residue. It could thus be deduced that the discovery of DMY with novel binding site of IKK-β could be useful for patients harboring gene mutation on IKK-β, especially on Cys-179 and ATP-binding regions (Phe-26 or F26).

Since IKK-β plays a vital role in the regulation of NF-κB signaling pathway which in turn leads to the regulation of transcription of genes involved in important mechanisms within cells such as T-cell activation, the medicinal usages thereof have been widely studied and published. For instance, IKK-β inhibitors have been proven to treat auto-immune diseases [Refs. 1-2], rheumatoid arthritis [Refs. 3-12], chronic obstructive pulmonary disease (COPD) and asthma [Refs. 11-27], cancer [Refs. 28-38], and diabetes [Refs. 39-42]. The references cited for each of the foregoing and hereinafter diseases in square bracket with “[Refs.xx]” with xx referring to the number of the corresponding literatures on the “References” list.

It can be deduced from the present invention that a compound or therapeutic agent that binds to cysteine-46 residue of IKK-β and inhibits the kinase activity of IKK-β can be used as inhibitors of IKK-β and NF-κB. As such, it can be further deduced by one skilled in the art that the aforesaid compound or therapeutic agent can be used for the treatment for the diseases described above as these diseases are associated with the activation of IKK-β and NF-κB.

In addition, NF-κB activation could mediate the Abeta-associated phenotype in Alzheimer disease, suggests the critical role in neurodegenerative diseases [Ref. 44]

It can also be deduced from the present invention that a compound or therapeutic agent that binds to cysteine-46 residue of IKK-β and inhibits the kinase activity of IKK-β can be used as suppressor of IKK-β/NF-κB activation. As such, it can be deduced by one skilled in the art that the aforesaid compound or therapeutic agent can be used for the treatment for the diseases described above as these diseases are associated with the activation of NF-κB signaling.

Further, it can be deduced from the present invention that a compound or therapeutic agent that binds to cysteine-46 residue of IKK-β and inhibits the kinase activity of IKK-β can be used as suppressor of immune reaction and hypersensitivity. As such, it can be deduced by one skilled in the art that the aforesaid compound or therapeutic agent can be used for the treatment for the diseases described above as these diseases are associated with the activation of immune reaction and hypersensitivity.

It can be deduced from the present invention that a compound or therapeutic agent that binds to cysteine-46 residue of IKK-β and inhibits the kinase activity of IKK-β can be used as inhibitor of arthritis. As such, it can be deduced by one skilled in the art that the aforesaid compound or therapeutic agent can be used for the treatment for the diseases described above as these diseases are associated with arthritis.

The present invention is further defined by the following examples, which are not intended to limit the present invention. Reasonable variations, such as those understood by reasonable artisans, can be made without departing from the scope of the present invention.

### Example 1

### Site-Directed Mutagenesis Assay

This example describes the assays that the cysteine or phenylalanine residue was mutated to alanine or one by one to establish the technique platform.

Cloning and Expression

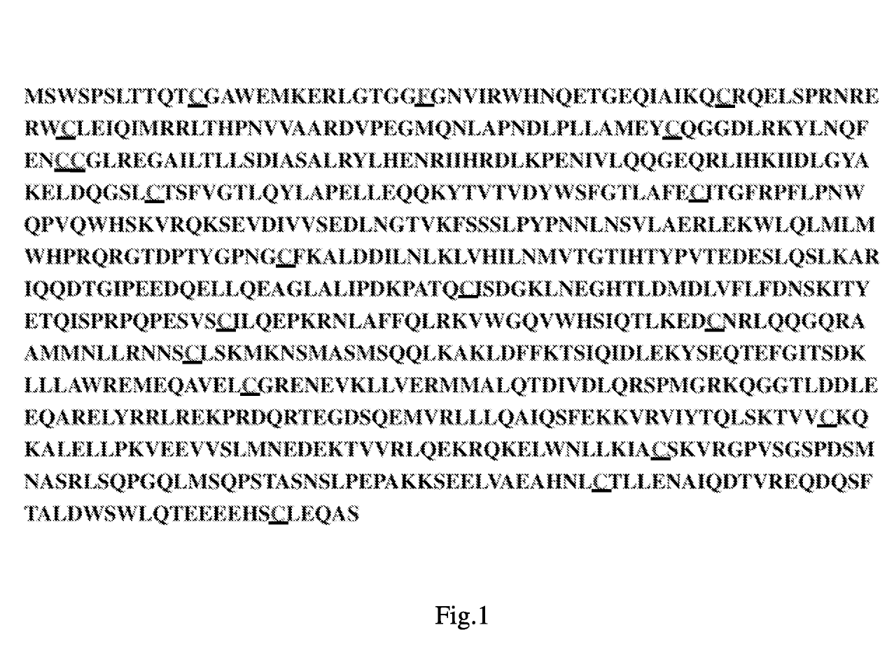

The FLAG-IKK-β construct was used as a template to introduce the single point mutants having cysteine (C) residue or phenylalanine (F) residue replaced with alanine (A) including C12A, C46A, C59A, C99A, C114A, C115A, C215A, C299A, C370A, C412A, C444A, C464A, C524A, C618A, C751A and F26A mutations. These mutated residues are underlined in the amino acid sequence (SEQ ID NO: 1) as shown in FIG. 1.

The site-directed mutagenesis was carried out using the Stratagene Quikchange Mutagenesis Kit accordingly to manufacturer's instructions. The mutations of clones were confirmed by DNA sequencing.

### Example 2

### Synthesis of Biotinylated DMY Assay, NF-κB Luciferase Reporter Assay, and IKK-β Kinase Assay

This example describes the synthesis of biotinylated DMY and comparison of the actions of DMY and DMY-biotin on T cell proliferation, NF-κB activation as well as IKK-β activity.

Synthesis of the Biotinylated DMY (DMY-Biotin)

Biotin (24.4 mg, 0.1 mmol) was suspended in dimethylformamide/dichloromethane (1:1, 2 mL), and dicyclohexylcarbodiimide (20.6 mg, 0.1 mmol) was added. After stirring at 60° C. for 5 minutes, dimethylaminopyridine (12.2 mg, 0.1 mmol) and DMY (48 mg, 0.15 mmol) in dimethylformamide (0.5 mL) were added. After stirring overnight, the mixture was poured into water (50 mL), acidified with 3M HCl to pH 3.0, and then extracted with ethyl acetate (20 mL×3). The residue of the organic layer was subjected to silica gel chromatography (petroleum ether: acetone from 4:3 to 1:3) to afford the target product as a yellow solid (25.1 mg, 46%). Negative HR-ESI-MS: m/z 545.1203 [M-H]—(calculated for C₂₅H₂₅N₂O₁₀S: 545.1230).

T Cell Proliferation Assay

T lymphocyte proliferation was assessed by 5-bromo-2′-deoxy-uridine (BrdU) assay. In brief, the isolated human T lymphocytes (10⁵ cells/well) were cultured in triplicates in a 96-well flat-bottomed plate (Costar, Corning Incorporated, Corning, N.Y., USA) in 100 μl of RPMI 1640 medium supplemented with 10% FBS and then co-stimulated with P/I or OKT-3/CD28 antibodies in the presence or absence of DMY (10-100 μM for 72 h. 5-bromo-2′-deoxy-uridine (BrdU, Roche) was added to the cells 14 h before the end of stimulation at a final concentration of 10 μM. BrdU can be incorporated into the DNA of growing cells during the labeling period; the amount of BrdU incorporated into the DNA can be quantified as an indicator of cell proliferation. In this experiment, BrdU was determined by ELISA according to manufacturer's instruction.

NF-κB Luciferase Reporter Assay

Jurkat cells were transiently transfected with NF-κB reporter plasmid with lipofectamine LTX according to the manufacturer's instructions. After transfection, cells were co-stimulated with P/I in the absence or presence of DMY or DMY-biotin for 6 h. Cellular proteins were lysed in Passive Lysis Buffer (Promega, Madison, Wis.). The transcriptional activity was determined by measuring the activity of firefly luciferase in a multi-well plate luminometer (Tecan, Durham, N.C.) using Luciferase Reporter Assay System (Promega).

IKK-β Kinase Assay

Anti-FLAG precipitated from HEK 293 expressing FLAG-IKK-β wt, as well as the GST-IκB-α substrate and ATP/Mg2Cl2 were incubated in the presence or absence of DMY or DMY-biotin for 1 h on ice. All of the entire components were analyzed by 10% SDS-PAGE. After electrophoresis, proteins were electro-transferred to the nitrocellulose membranes. After the transfer, the membranes were blocked by 5% dried milk for 60 min and then washed three times (5 min in each wash) with TBS-T. The membranes were incubated with P-IκBα antibodies overnight at 4° C. and then washed three times with TBS-T. Afterwards, the membranes were incubated again with HRP-conjugated secondary antibodies for 60 min The blots were developed using the ECL.

Results

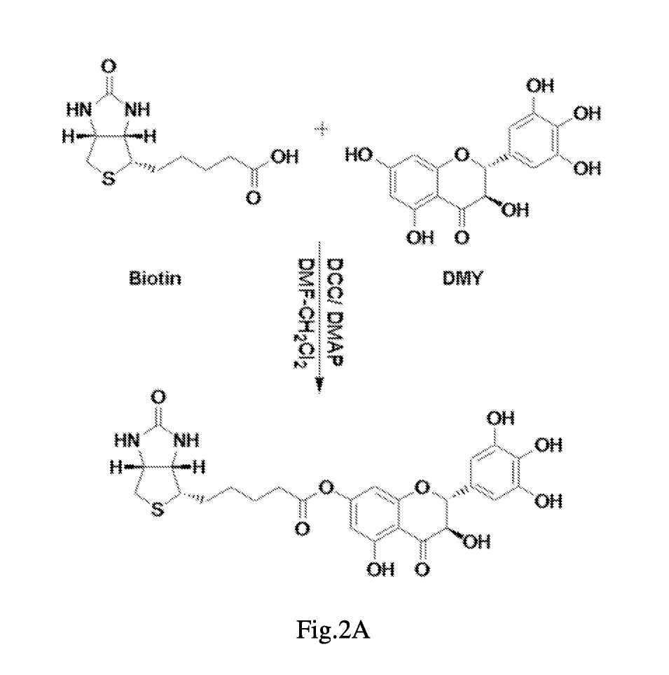

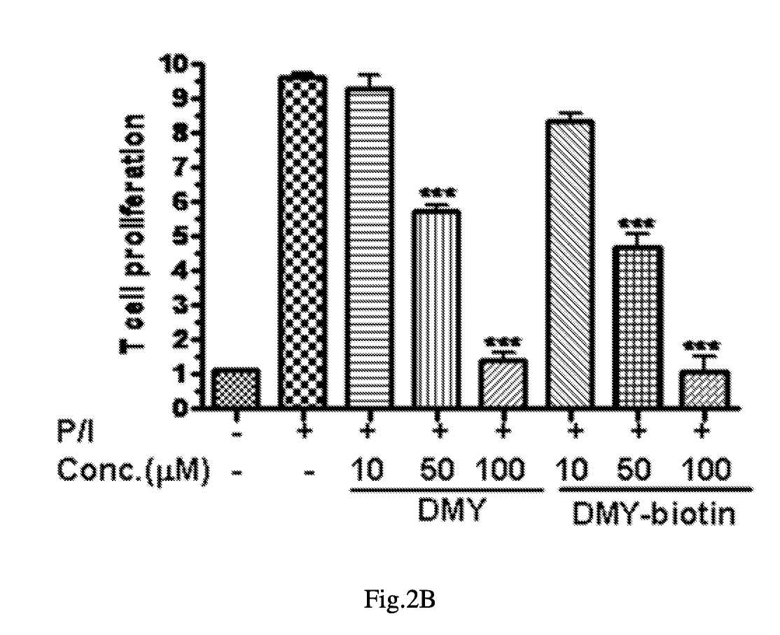

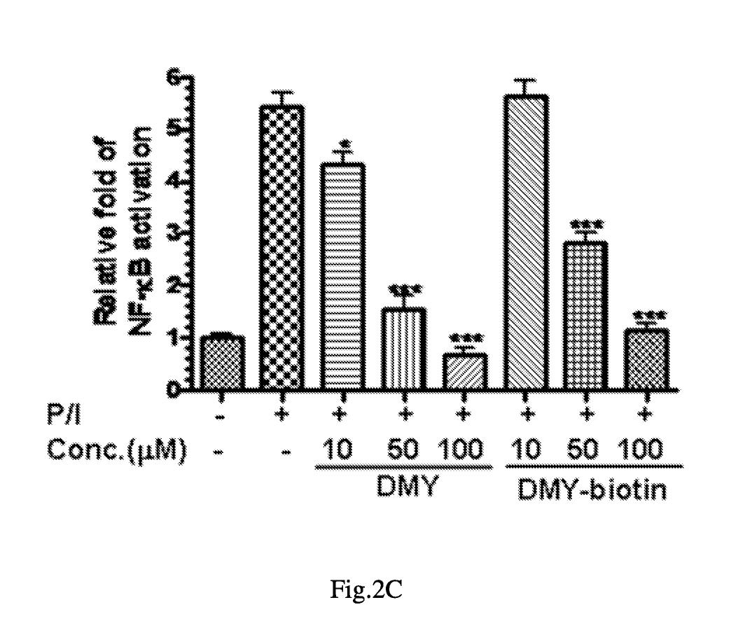

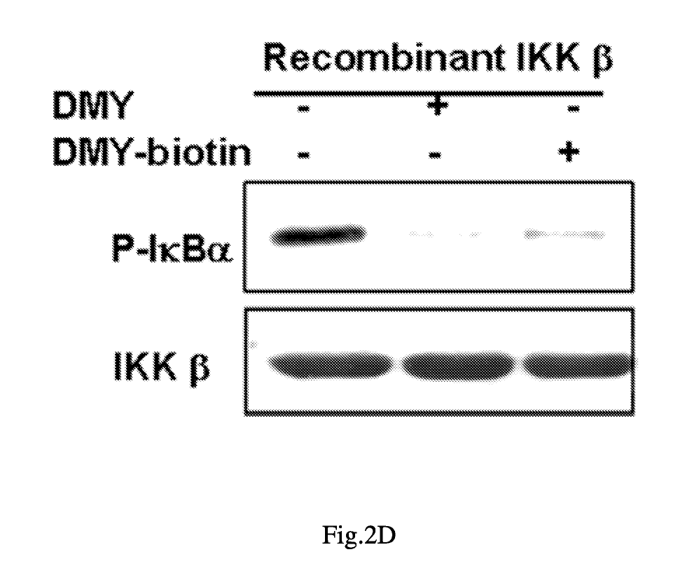

It can be observed from FIGS. 2B to 2D that DMY and DMY-biotin can inhibit T cell proliferation (FIG. 2B), NF-κB activation (FIG. 2C), as well as IKK-β kinase activity (FIG. 2D).

### Example 3

### Study on Binding Sites of IKK-β for DMY and DMY-Biotin

This example describes the assay to show that DMY directly binds to IKK-β using DMY-biotin probe; further, DMY-biotin and DMY compound are shown to share the same binding site on IKK-β.

IKK-β Competition Assay

20 ng of human recombinant IKK-β was incubated with 100 μM of the DMY-biotin in the presence of 0, 1 and 5 folds of concentration of its parental compound DMY. The mixture was dropped on the nitrocellulose membranes, and then detected with streptavidin horseradish peroxidase (Sigma). The binding signal was then detected by using ECL.

Results

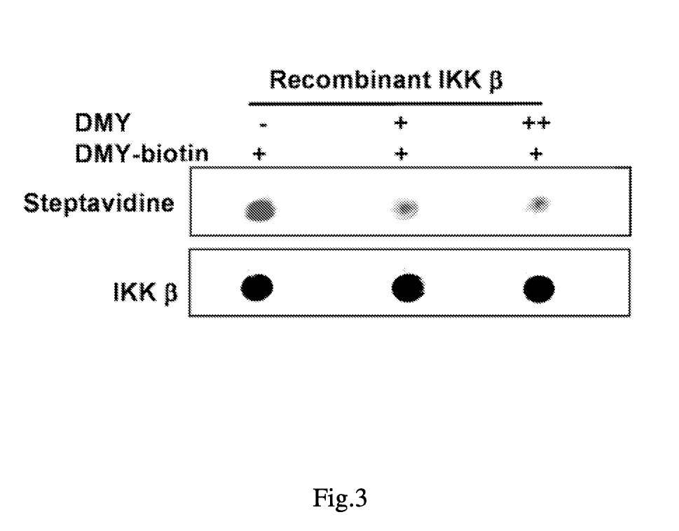

As illustrated in FIG. 3, the assay shows that the parental compound DMY can compete with the biotin-DMY, indicating that the DMY-biotin is confirmed to exhibit an identical binding site(s) as its parental compound DMY.

### Example 4

### Study on Novel Binding Site(s) of IKK-β for DMY

This example describes the assays to show that the binding site of DMY-biotin on IKK-β is novel rather than known drug binding site(s), e.g. ATP binding site, Cys-179, Ser-177/181 and allosteric binding site.

IKK-β Displacement Binding Assay

Anti-FLAG precipitated from HEK 293 expressing FLAG-IKK-β was incubated with Berberine, BMS-345541, SC-514 and BOT-64 for 1 h on ice, and then the mixture were incubated with 100 μM DMY-biotin. Subsequently, the proteins were separated by SDS-PAGE and transferred to nitrocellulose membranes. After blocking with BSA and washing with PBS-T (Tween-20, 0.05%), the membranes were incubated with streptavidin horseradish peroxidase (Sigma) and developed with ECL.

Results

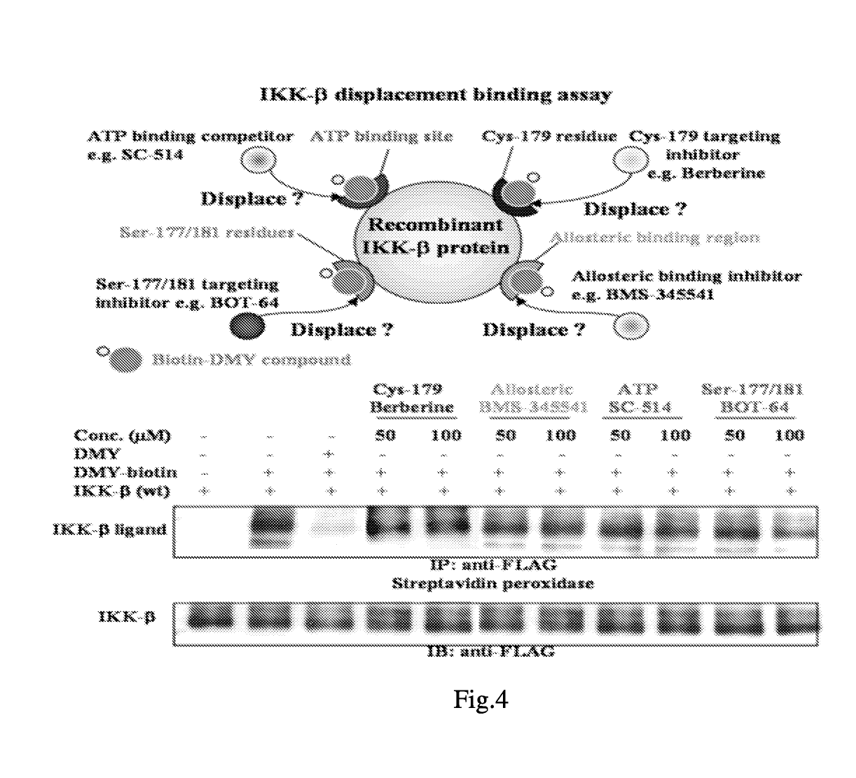

As shown in FIG. 4, DMY binds to IKK-β protein via novel but not well-known binding site(s).

### Example 5

### Study on Effect of DMY on Drug Resistant Phenotype of IKK-β Mutants

This example describes the assay to show that DMY is able to circumvent the drug resistant phenotype of IKK-β mutants with Cys-179 (C179A) and ATP-binding site (F26A) mutations.

IKK-β Kinase Assay

Anti-FLAG precipitated from HEK 293 expressing FLAG-IKK-β C179A, F26A, as well as the GST-IκB-α substrate and ATP/Mg2C12 were incubated with or without DMY for 1 h on ice. All of the components were analyzed by 10% SDS-PAGE. After electrophoresis, the proteins were electro-transferred to the nitrocellulose membranes. After the transfer, the membranes were blocked by 5% dried milk for 60 min and then washed three times (5 min in each wash) with TBS-T. The membranes were incubated with P-IκBα antibodies overnight at 4° C. and then washed three times with TBS-T. Afterwards, the membranes were incubated again with HRP-conjugated secondary antibodies for 60 min. The blots were developed using the ECL.

IKK-β Mutant Kinase Assay

Anti-FLAG precipitated from HEK 293 expressing FLAG-IKK-β C179A or F26A were incubated with DMY for 1 h on ice, and then separated by SDS-PAGE and transferred to nitrocellulose membranes. After blocking with BSA and washing with PBS-T (Tween-20, 0.05%), the membranes were incubated with streptavidin horseradish peroxidase (Sigma) and developed with ECL.

Results

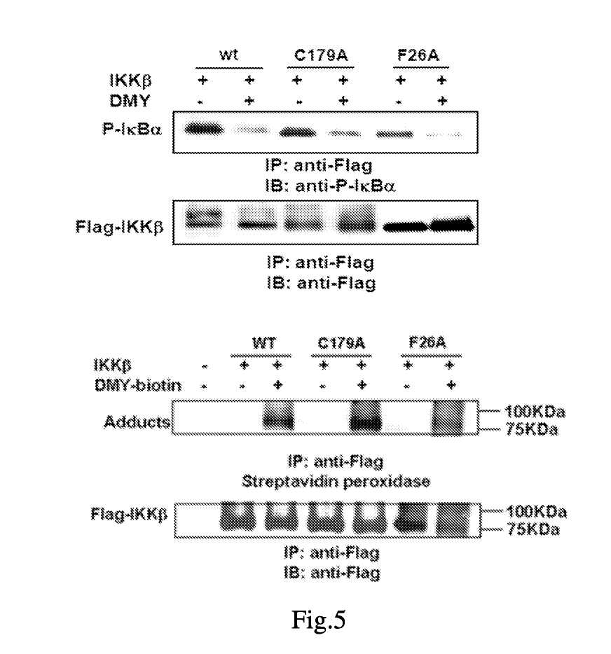

As illustrated in FIG. 5, DMY was shown to circumvent the drug resistant phenotype of IKK-β mutants with Cys-179 (C179A) and ATP-binding site (F26A) mutations. Hence, DMY was shown to bind to IKK-β via binding site(s) other than the well-known binding sites of Cys-179 residue and ATP-binding site (Phe-26).

### Example 6

Study on Effect of DMY on IKK-β with Cysteine-46 Mutation (C46A)

This example describes the assay to show that DMY fails to suppress the kinase activity of IKK-β mutant with cysteine-46 mutation (C46A), as well as form protein adduct with IKK-β mutant (C46A).

IKK-β Kinase Assay

Anti-FLAG precipitated from HEK 293 expressing FLAG-IKK-β C46A, as well as the GST-IκB-α substrate and ATP/Mg₂Cl₂ were incubated with or without DMY for 1 h on ice. All of the components were analyzed by 10% SDS-PAGE. After electrophoresis, the proteins were electro-transferred to the nitrocellulose membranes. After the transfer, the membranes were blocked by 5% dried milk for 60 min and then washed three times (5 min in each wash) with TBS-T. The membranes were incubated with P-IκBα antibodies overnight at 4° C. and then washed three times with TBS-T. Afterwards, the membranes were incubated again with HRP-conjugated secondary antibodies for 60 min. The blots were developed using the ECL.

IKK-β Mutant Kinase Assay

Anti-FLAG precipitated from HEK 293 expressing FLAG-IKK-β C46A were incubated with DMY for 1 h on ice, and then separated by SDS-PAGE and transferred to nitrocellulose membranes. After blocking with BSA and washing with PBS-T (Tween-20, 0.05%), the membranes were incubated with streptavidin horseradish peroxidase (Sigma) and developed with ECL.

Results

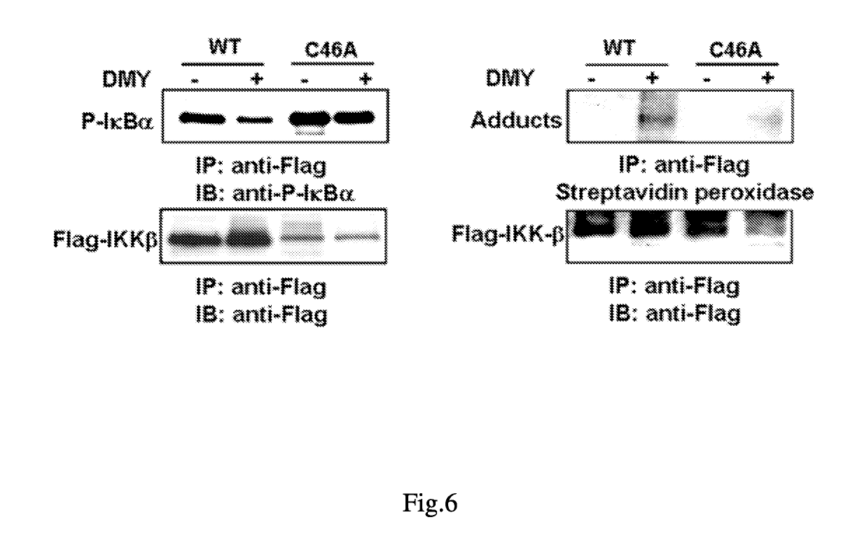

As seen from FIG. 6, DMY cannot inhibit the kinase activity of IKK-β mutant with cysteine-46 mutation (C46A), nor form protein adduct with IKK-β mutant (C46A). Hence, DMY was shown to bind to C46 residue of IKK-β.

### Example 7

### Study on Effect of DMY on IKK-β with Cysteine Mutations

This example describes the assay to show that DMY is able to suppress IKK-β mutants with cysteine mutations of C12A, C59A, C99A, C114A, C115A, C215A, C299A, C370A, C412A, C444A, C464A, C524A, C618A, C662/716A and C751A mutations.

IKK-β Kinase Assay

Anti-FLAG precipitated from HEK 293 expressing FLAG-IKK-β wild-type (wt) or mutants with cysteine mutations of C12A, C59A, C99A, C114A, C115A, C215A, C299A, C370A, C412A, C444A, C464A, C524A, C618A, C662/716A and C751A mutations as well as the GST-IκB-α substrate and ATP/Mg₂Cl₂ were incubated with or without DMY for 1 h on ice. All of the components were analyzed by 10% SDS-PAGE. After electrophoresis, the proteins were electro-transferred to the nitrocellulose membranes. After the transfer, the membranes were blocked by 5% dried milk for 60 min and then washed three times (5 min in each wash) with TBS-T. The membranes were incubated with P-IκBα antibodies overnight at 4° C. and then washed three times with TBS-T. Afterwards, the membranes were incubated again with HRP-conjugated secondary antibodies for 60 min. The blots were developed using the ECL.

Results

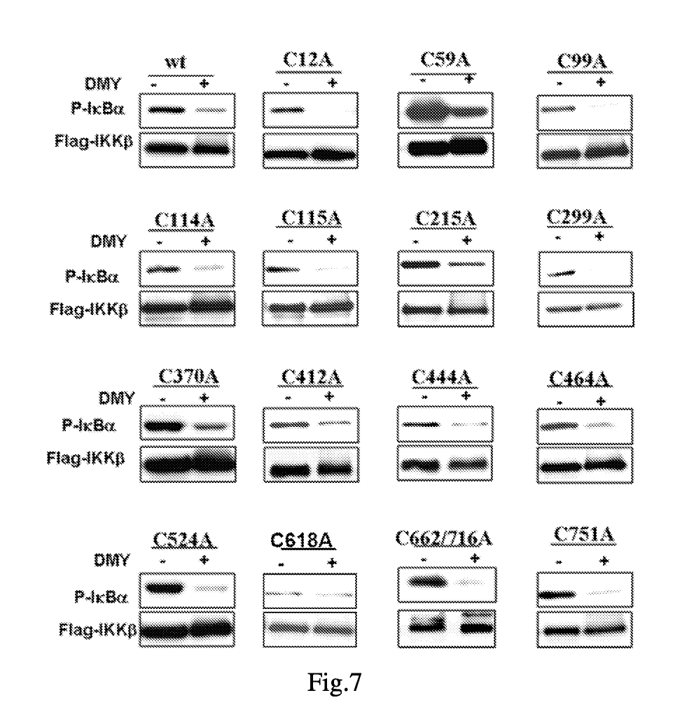

As shown in FIG. 7, DMY is able to suppress IKK-β mutants with cysteine mutations of C12A, C59A, C99A, C114A, C115A, C215A, C299A, C370A, C412A, C444A, C464A, C524A, C618A, C662/716A and C751A mutations. Hence, DMY was shown not to bind to C12 residue, C59 residue, C99 residue, C114 residue, C115 residue, C179 residue, C215 residue, C299 residue, C370 residue, C412 residue, C444 residue, C464 residue, C524 residue, C618 residue, C662/C716 residue and C751 residue of IKK-β.

### Example 8

### Study on Formation of Protein Adduct from DMY and IKK-β with Cysteine Mutations

This example describes the assay to show that DMY is able to form protein adduct with IKK-β mutants with cysteine mutations of C12A, C59A, C99A, C114A, C115A, C215A, C299A, C370A, C412A, C444A, C464A, C524A, C618A, C662/716A and C751A mutations.

Protein Adduct Formation Assay

Anti-FLAG precipitated from HEK 293 expressing FLAG-IKK-β wild-type (wt) or mutants with cysteine mutations of C12A, C59A, C99A, C114A, C115A, C215A, C299A, C370A, C412A, C444A, C464A, C524A, C618A, C662/716A and C751A mutations, were incubated with DMY for 1 h on ice, and then separated by SDS-PAGE and transferred to nitrocellulose membranes. After blocking with BSA and washing with PBS-T (Tween-20, 0.05%), the membranes were incubated with streptavidin horseradish peroxidase (Sigma) and developed with ECL.

Results

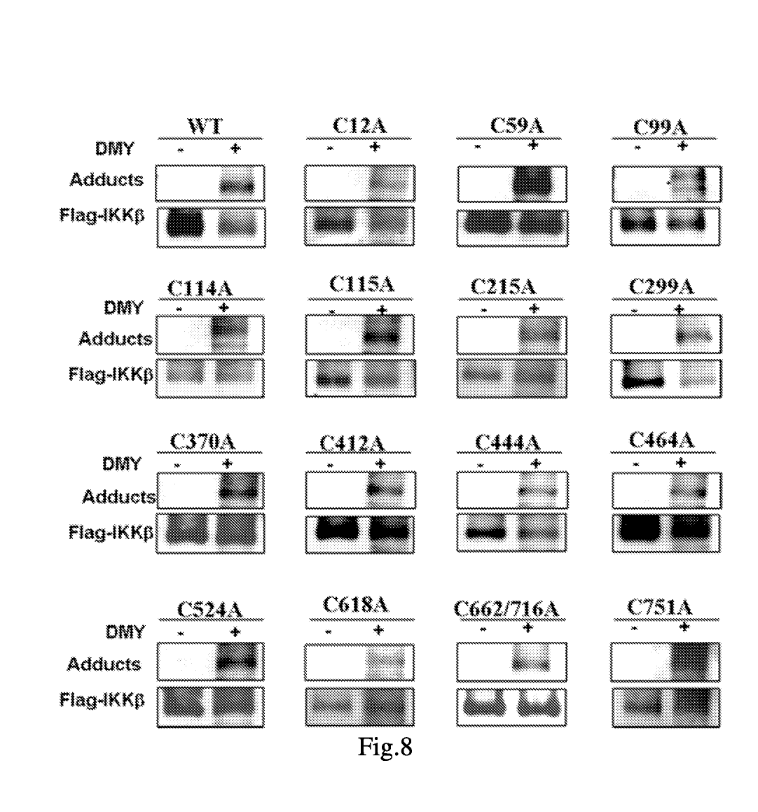

As shown in FIG. 8, DMY formed protein adduct with IKK-β mutants with cysteine mutations, i.e. C12A, C59A, C99A, C114A, C115A, C215A, C299A, C370A, C412A, C444A, C464A, C524A, C618A, C662/C716A and C751A. Hence, DMY was shown not to bind nor form protein adduct with C12 residue, C59 residue, C99 residue, C114 residue, C115 residue, C215 residue, C299 residue, C370 residue, C412 residue, C444 residue, C464 residue, C524 residue, C618 residue, C662/716 residue and C751 residue of IKK-β.

### Example 9

### Study on Effect of DMY to Suppress IKK-β-NF-κB Signaling

This example describes that DMY is able to suppress IKK-β-NF-κB signaling through Cys-46 residue of IKK-β in a cellular model.

Evaluation in Cellular Model

IKK-β−/− deficient MEFs transfected with FLAG-IKK-β (wt) plasmid or mutant FLAG-IKK-β (C46A) plasmid were pretreated with or without 50 μM DMY, followed by treatment of 20 ng/mL of TNF-α. The MEFs lysates were prepared for Western blotting analysis using antibodies against phosphorylation of NF-κB p65 and IκBα.

Results

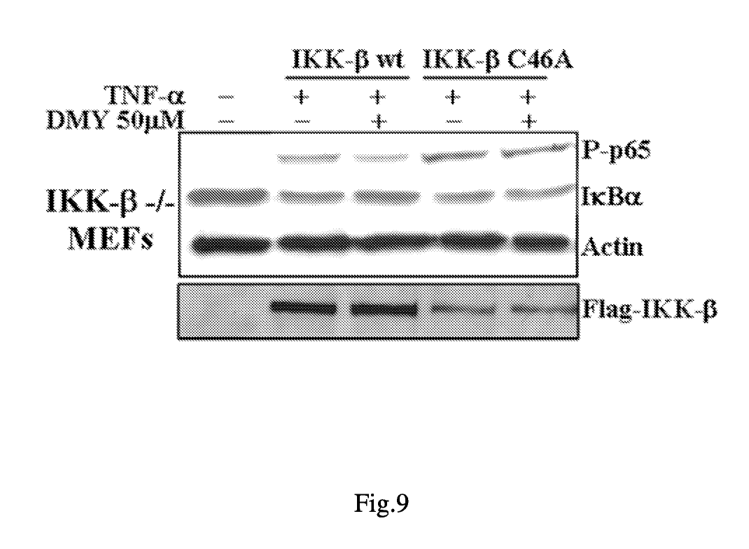

As shown in FIG. 9, DMY cannot suppress IKK-β-NF-κB signaling through Cys-46 residue of IKK-β mutant (C46A) in IKK-β deficient cells model.

### Example 10

### Study on Ear Edema

The example describes the assays to show that topically application of DMY is effective to relief mouse ear edema.

The delay-type hypersensitivity test (DTHT) in mice

Male ICR mice, weighting 22-30 g, were obtained from the Laboratory Animal Services Center, the Chinese University of Hong Kong (Hong Kong, China). Male mice were sensitized through topical application of 20 μl of 0.5% (v/v) dinitrofluorobenzene (DNFB) in acetone onto the shaved abdomen on days 1 and 2. Challenge was then preformed in day 6 by applying DNFB (20 μl, 0.5%, v/v) on the left inner and outer ear surfaces of mice. DMY (at doses of 0.5, 1, 2 mg/ear) and DEX (0.025 mg/ear, Sigma-Aldrich) dissolved in acetone was topically applied (20 μl) to the ears at 2nd, 24th, 48th, and 72nd hour after the challenge. The mice were sacrificed by cervical dislocation, and then the same area of the ears was punched from each animal Spleens and thymuses were isolated and weighted. The ear edema was calculated according to the differences between the weight of the right and left ears. The control group was treated only with DNFB.

Results

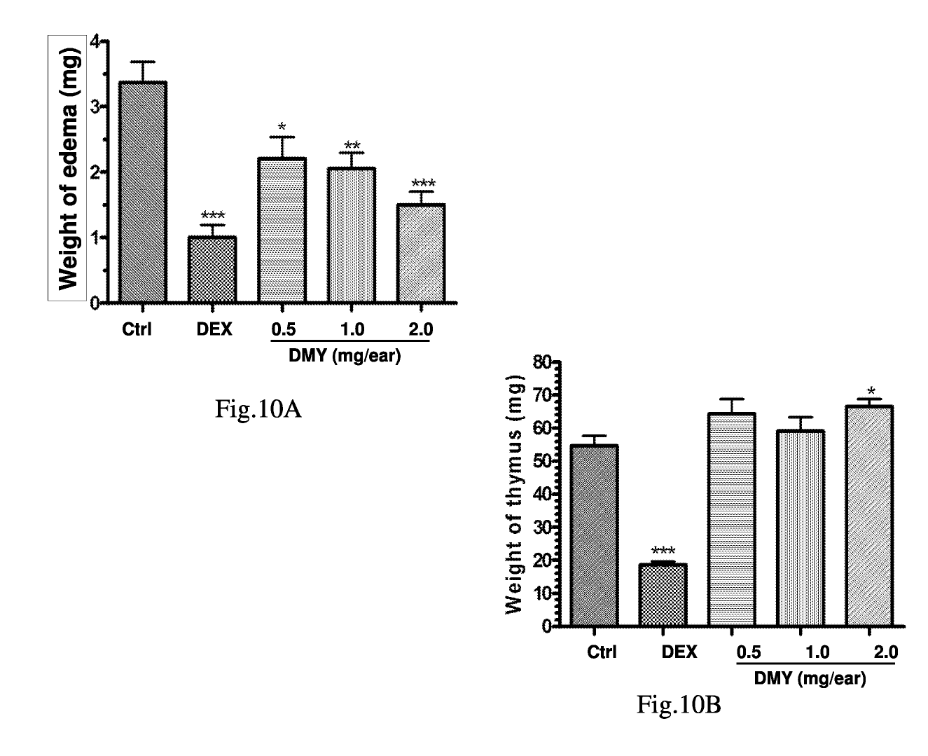

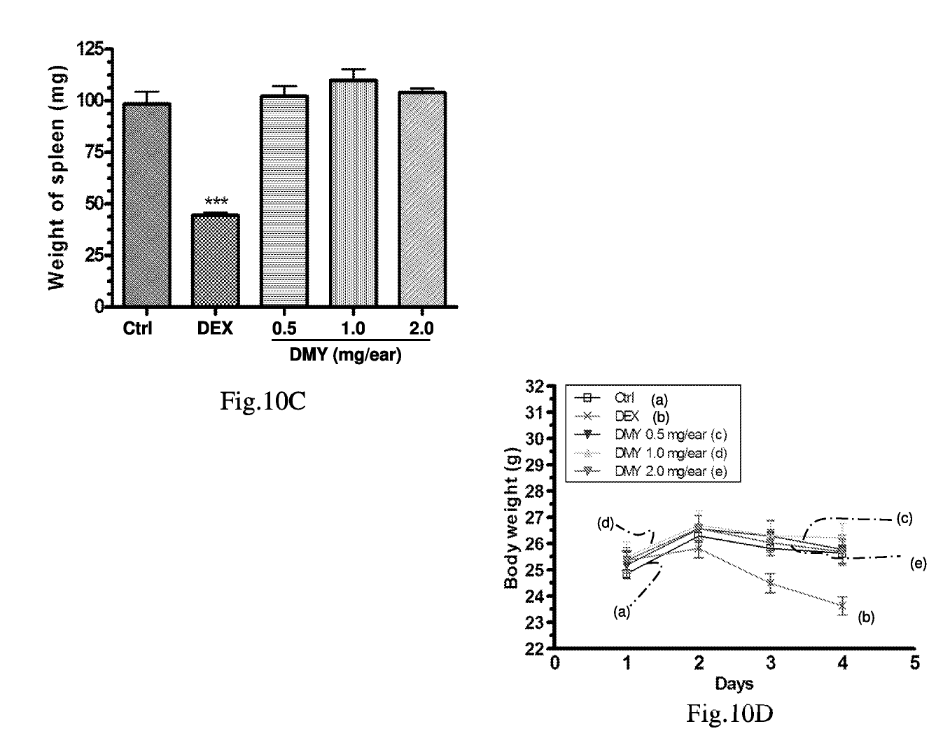

The DTHT test is the reaction triggered by antigen-specific T cells that can be induced by different allergens. In this study, the most commonly used allergen, DNFB which can effectively induce the contact dermatitis on ears was used. As observed from FIG. 10A, DMY could significantly and dose-dependently inhibit the ear edema of mice and the inhibition induced by of DMY is similar to the effect of DEX.

Besides, from FIGS. 10B and C spleen and thymus weights of the mice were decreased for DEX treatment, whereas an increase of weights of spleen and thymus can be observed for DMY treatment. Further, the body weight of the mice was greatly reduced for DEX treatment, while only a small decrease of body weight can be observed for mice treated with DMY in which the differences between body weights of mice in DMY treatment group and the control group were not significant.

In view of the above results, DMY suppresses hypersensitivity reaction of mouse ear edema induced by DNFB. DMY is also proven to be efficacious for the treatment of dermatitis, ear inflammation, and general inflammation, without adverse effect of general immunity suppression.

### Example 11

### Study on Arthritis

This example describes the study to show that DMY is effective to ameliorate collagen II induced arthritis in rats.

The Collagen II Induced Arthritis (CIA) in Rats

Female Wistar rats, 5-6 weeks old, were obtained from the Laboratory Animal Services Center, the Chinese University of Hong Kong (Hong Kong, China). Collagen II solution (collagen, 2 mg/ml in 0.05M acetic acid, Chondrex 20022, Redmond, Wash., USA) was emulsified with an equal volume of incomplete Freund's adjuvant (IFA, Chondrex 7002, Redmond, Wash., USA) at 4° C. using a high-speed homogenizer. In the experiment of CIA, DMY was encapsulated with HP-CD (1:8.48) and then dissolved in the normal saline with drug concentrations of 50 and 100 mg/kg body weight. Rats were intradermally injected at the base of the tail with 100 μl collagen/incomplete Freund's adjuvant (IFA) emulsion containing 100 μg of collagen II by the use of a glass syringe equipped with a locking hub and a 27-G needle. On day 7 after the primary immunization, all the rats were given a booster injection of 100 μg of collagen II in IFA. On the day after the onset of arthritis (day 13), the CIA rats were exposed to a daily intraperitoneal administration of DMY (50 and 100 mg/kg) until day 30 of the study. DEX (0.1 mg/kg, one per day), MTX (3.75 mg/kg, twice per week), and indomethacin (1 mg/kg, one per day) were used as positive reference drugs.

The rats were inspected daily from the onset of arthritis characterized by edema and/or erythema in the paws. The incidence and severity of arthritis were evaluated using an arthritic scoring system, and bi-hind paw volumes and body weight were measured every 2 days started on the day when the arthritic signs were firstly visible (day 13). In the arthritic scoring system, lesions (i.e., the clinical arthritic signs) of the four paws of each rat were graded from 0 to 4 according to the extent of both edema and erythema of the periarticular tissues. As such, 16 was the potential maximum of the combined arthritic scores per animal. The hind paw volumes were measured using a plethysmometer chamber (7140 UGO. Basile, Comerio, Italy) and expressed as the mean volume change of both hind paws of the rats. Body weight of the rats was monitored with a 0.1 g precision balance (Sartorius A G, Goettingen, Germany). On day 30, all rats were sacrificed with liver, spleen and thymus being collected and weighted. The organ index for a specific organ is equal to the ratio of the weight of that organ to a body weight of 100 g.

Results

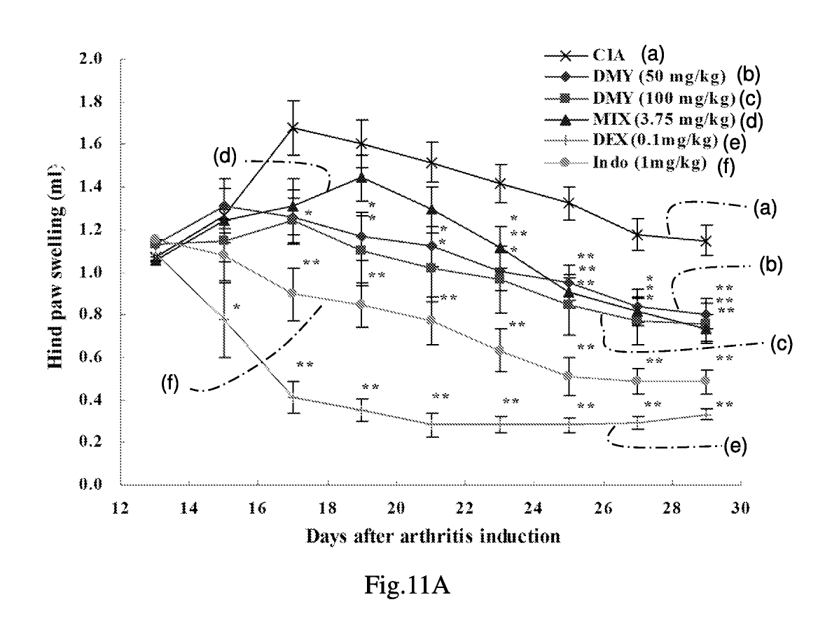

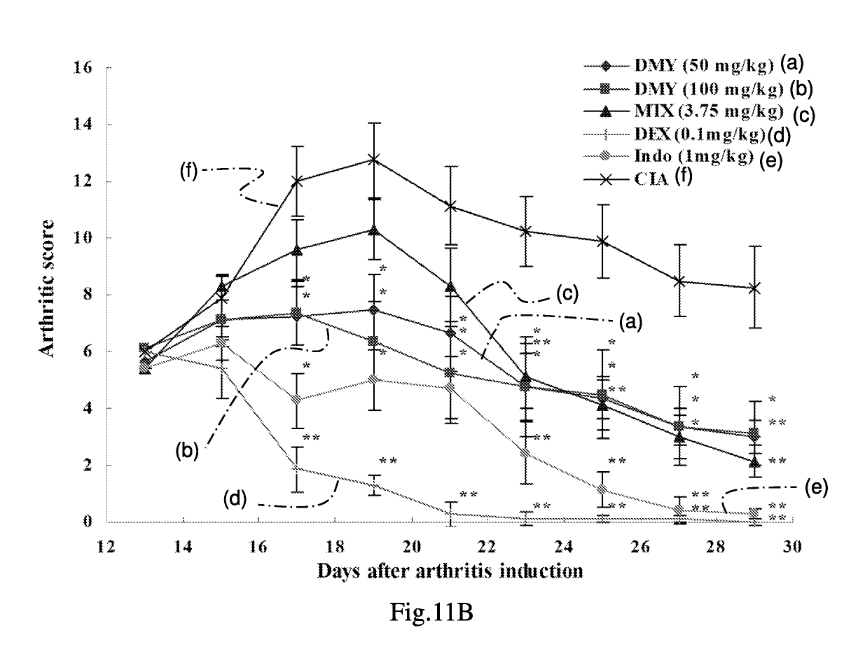

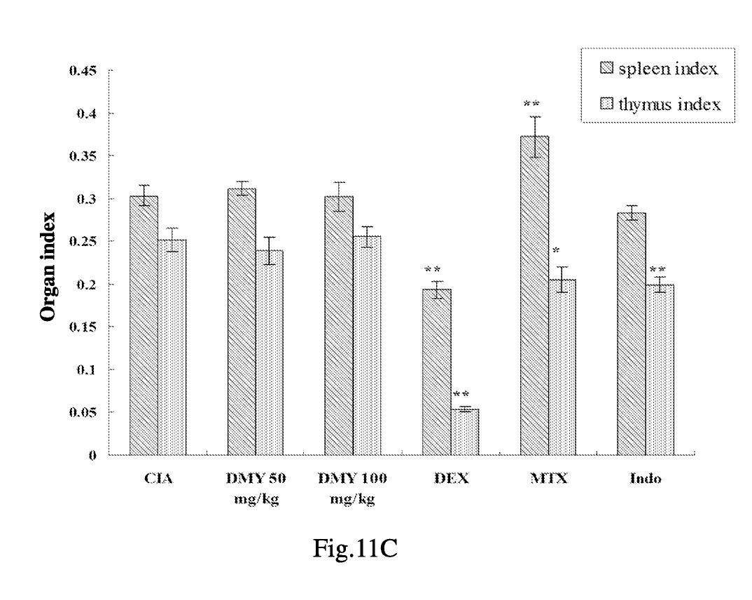

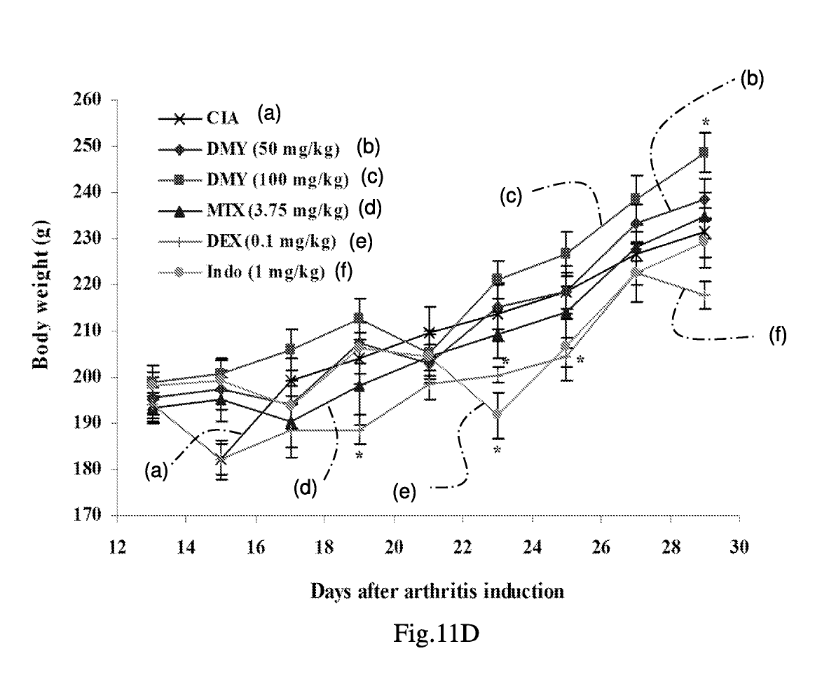

From FIGS. 11A and B, DMY treatment significantly reduced both the hind paw volume and the arthritic scores as compared to those of the vehicle-treated CIA rats, and the ameliorative effect of DMY at dose of 100 mg/kg (equivalent to human dose 16 mg/kg) was shown to be better than that of MTX. More importantly, it can be seen from FIG. 11C that there was no adverse effect on the organ indexes of spleen and thymus for DMY treatment, whereas treatments with DEX, MTX, or indomethacin led to a significant reduction of the organ indexes of spleen and/or thymus. In addition, a significant reduction in body weight can be observed for DEX-, MTX-, or indomethacin-treated animals from FIG. 11D, while the DMY-treated rats were shown even to have increase of the body weight.

In view of the above results, DMY suppresses arthritis induced by collagen II in rats. DMY is also proven to be efficacious for the treatment of arthritis and thus inflammation without adverse effect of general immunity suppression. The use of DMY is as described in the previous example.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

### REFERENCES

1. Keifer J A, Guttridge D C, Ashburner B P, Baldwin A S, Jr. Inhibition of NF-kappa B activity by thalidomide through suppression of IkappaB kinase activity. J Biol Chem 2001; 276(25):22382-7.
2. Rossi A, Kapahi P, Natoli G, et al. Anti-inflammatory cyclopentenone prostaglandins are direct inhibitors of IkappaB kinase. Nature 2000; 403(6765):103-8.
3. Hammaker D, Sweeney S, Firestein GS. Signal transduction networks in rheumatoid arthritis. Ann Rheum Dis 2003; 62 Suppl 2:ii86-9.
4. Castro A C, Dang L C, Soucy F, et al. Novel IKK inhibitors: beta-carbolines. Bioorg Med Chem Lett 2003; 13(14):2419-22.
5. Kishore N, Sommers C, Mathialagan S, et al. A selective IKK-2 inhibitor blocks NF-kappa B-dependent gene expression in interleukin-1 beta-stimulated synovial fibroblasts. J Biol Chem 2003; 278(35):32861-71.
6. Nagashima K, Sasseville V G, Wen D, et al. Rapid TNFR1-dependent lymphocyte depletion in vivo with a selective chemical inhibitor of IKKbeta. Blood 2006; 107(11):4266-73.
7. Schopf L, Savinainen A, Anderson K, et al. IKKbeta inhibition protects against bone and cartilage destruction in a rat model of rheumatoid arthritis. Arthritis Rheum 2006; 54(10):3163-73.
8. Burke J R, Pattoli M A, Gregor K R, et al. BMS-345541 is a highly selective inhibitor of I kappa B kinase that binds at an allosteric site of the enzyme and blocks NF-kappa B-dependent transcription in mice. J Biol Chem 2003; 278(3):1450-6.
9. McIntyre K W, Shuster D J, Gillooly K M, et al. A highly selective inhibitor of I kappa B kinase, BMS-345541, blocks both joint inflammation and destruction in collagen-induced arthritis in mice. Arthritis Rheum 2003; 48(9):2652-9.
10. MacMaster J F, Dambach D M, Lee D B, et al. An inhibitor of IkappaB kinase, BMS-345541, blocks endothelial cell adhesion molecule expression and reduces the severity of dextran sulfate sodium-induced colitis in mice. Inflamm Res 2003; 52(12):508-11.
11. Karin M, Yamamoto Y, Wang Q M. The IKK NF-kappa B system: a treasure trove for drug development. Nat Rev Drug Discov 2004; 3(1):17-26.
12. Ziegelbauer K, Gantner F, Lukacs N W, et al. A selective novel low-molecular-weight inhibitor of IkappaB kinase-beta (IKK-beta) prevents pulmonary inflammation and shows broad anti-inflammatory activity. Br J Pharmacol 2005; 145(2):178-92.
13. Bamborough P, Callahan J F, Christopher J A, et al. Progress towards the development of anti-inflammatory inhibitors of IKKbeta. Curr Top Med Chem 2009; 9(7):623-39.
14. Edwards M R, Bartlett N R, Clarke D, Birrell M, Belvisi M, Johnston S L. Targeting the NF-kappaB pathway in asthma and chronic obstructive pulmonary disease. Pharmacol Ther 2009; 121(1):1-13.
15. Gagliardo R, Chanez P, Mathieu M, et al. Persistent activation of nuclear factor-kappaB signaling pathway in severe uncontrolled asthma. Am J Respir Crit. Care Med 2003; 168(10):1190-8.
16. La Grutta S, Gagliardo R, Mirabella F, et al. Clinical and biological heterogeneity in children with moderate asthma. Am J Respir Crit. Care Med 2003; 167(11):1490-5.
17. Caramori G, Romagnoli M, Casolari P, et al. Nuclear localisation of p65 in sputum macrophages but not in sputum neutrophils during COPD exacerbations. Thorax 2003; 58(4):348-51.
18. Di Stefano A, Caramori G, Oates T, et al. Increased expression of nuclear factor-kappaB in bronchial biopsies from smokers and patients with COPD. Eur Respir J 2002; 20(3):556-63.
19. Sadikot R T, Zeng H, Joo M, et al. Targeted immunomodulation of the NF-kappaB pathway in airway epithelium impacts host defense against Pseudomonas aeruginosa. J Immunol 2006; 176(8):4923-30.
20. Podolin P L, Callahan J F, Bolognese B J, et al. Attenuation of murine collagen-induced arthritis by a novel, potent, selective small molecule inhibitor of IkappaB Kinase 2, TPCA-1 (2-[(aminocarbonyl)amino]-5-(4-fluorophenyl)-3-thiophenecarboxamide), occurs via reduction of proinflammatory cytokines and antigen-induced T cell Proliferation. J Pharmacol Exp Ther 2005; 312(1):373-81.
21. Hideshima T, Chauhan D, Richardson P, et al. NF-kappa B as a therapeutic target in multiple myeloma. J Biol Chem 2002; 277(19):16639-47.
22. Wen D, Nong Y, Morgan J G, et al. A selective small molecule IkappaB Kinase beta inhibitor blocks nuclear factor kappaB-mediated inflammatory responses in human fibroblast-like synoviocytes, chondrocytes, and mast cells. J Pharmacol Exp Ther 2006; 317(3):989-1001.
23. Onai Y, Suzuki J, Kakuta T, et al. Inhibition of IkappaB phosphorylation in cardiomyocytes attenuates myocardial ischemia/reperfusion injury. Cardiovasc Res 2004; 63(1):51-9.
24. Pierce J W, Schoenleber R, Jesmok G, et al. Novel inhibitors of cytokine-induced IkappaBalpha phosphorylation and endothelial cell adhesion molecule expression show anti-inflammatory effects in vivo. J Biol Chem 1997; 272(34):21096-103.
25. Frelin C, Imbert V, Griessinger E, Loubat A, Dreano M, Peyron J F. AS602868, a pharmacological inhibitor of IKK2, reveals the apoptotic potential of TNF-alpha in Jurkat leukemic cells. Oncogene 2003; 22(50):8187-94.
26. Birrell M A, Hardaker E, Wong S, et al. Ikappa-B kinase-2 inhibitor blocks inflammation in human airway smooth muscle and a rat model of asthma. Am J Respir Crit. Care Med 2005; 172(8):962-71.
27. Chapoval S P, Al-Garawi A, Lora J M, et al Inhibition of NF-kappaB activation reduces the tissue effects of transgenic IL-13. J Immunol 2007; 179(10):7030-41.
28. Hu M C, Lee D F, Xia W, et al. IkappaB kinase promotes tumorigenesis through inhibition of forkhead FOXO3a. Cell 2004; 117(2):225-37.
29. Gringhuis S I, Garcia-Vallejo J J, van Het Hof B, van Dijk W. Convergent actions of I kappa B kinase beta and protein kinase C delta modulate mRNA stability through phosphorylation of 14-3-3 beta complexed with tristetraprolin. Mol Cell Biol 2005; 25(15):6454-63.
30. Lee S, Andrieu C, Saltel F, et al. IkappaB kinase beta phosphorylates Dok1 serines in response to TNF, IL-1, or gamma radiation. Proc Natl Acad Sci USA 2004; 101(50):17416-21.
31. Lee D F, Kuo H P, Chen C T, et al. IKK beta suppression of TSC1 links inflammation and tumor angiogenesis via the mTOR pathway. Cell 2007; 130(3):440-55.
32. Xia Y, Padre R C, De Mendoza T H, Bottero V, Tergaonkar V B, Verma I M. Phosphorylation of p53 by IkappaB kinase 2 promotes its degradation by beta-TrCP. Proc Natl Acad Sci USA 2009; 106(8):2629-34.
33. Yin M J, Yamamoto Y, Gaynor R B. The anti-inflammatory agents aspirin and salicylate inhibit the activity of I(kappa)B kinase-beta. Nature 1998; 396(6706):77-80.
34. Yamamoto Y, Yin M J, Lin K M, Gaynor R B. Sulindac inhibits activation of the NF-kappaB pathway. J Biol Chem 1999; 274(38):27307-14.
35. Waxman S, Anderson K C. History of the development of arsenic derivatives in cancer therapy. Oncologist 2001; 6 Suppl 2:3-10.
36. Kapahi P, Takahashi T, Natoli G, et al Inhibition of NF-kappa B activation by arsenite through reaction with a critical cysteine in the activation loop of Ikappa B kinase. Biol Chem 2000; 275(46):36062-6.
37. Reynaert N L, van der Vliet A, Guala A S, et al. Dynamic redox control of NF-kappaB through glutaredoxin-regulated S-glutathionylation of inhibitory kappaB kinase beta. Proc Natl Acad Sci USA 2006; 103(35):13086-91.
38. Olsen L S, Hjarnaa P J, Latini S, et al. Anticancer agent CHS 828 suppresses nuclear factor-kappa B activity in cancer cells through downregulation of IKK activity. Int J Cancer 2004; 111(2):198-205.
39. Gao Z, Hwang D, Bataille F, et al. Serine phosphorylation of insulin receptor substrate 1 by inhibitor kappa B kinase complex. J Biol Chem 2002; 277(50):48115-21.
40. Nakamori Y, Emoto M, Fukuda N, et al. Myosin motor Myolc and its receptor NEMO/IKK-gamma promote TNF-alpha-induced serine-307 phosphorylation of IRS-1. J Cell Biol 2006; 173(5):665-71.
41. Kamon J, Yamauchi T, Muto S, et al. A novel IKKbeta inhibitor stimulates adiponectin levels and ameliorates obesity-linked insulin resistance. Biochem Biophys Res Commun 2004; 323(1):242-8.
42. Yuan M, Konstantopoulos N, Lee J, et al. Reversal of obesity- and diet-induced insulin resistance with salicylates or targeted disruption of Ikkbeta. Science 2001; 293(5535):1673-7.

What is claimed is:

1. A method of screening a therapeutic agent as a drug candidate for cancer, inflammation, neurodegenerative disease, immunological disorder, or arthritic disorder comprising:
a) exposing said agent to an assay comprising IKK-β;
b) detecting whether said agent binds to cysteine-46 residue of IKK-β;
c) detecting whether said agent inhibits kinase activity of IKK-β upon said binding in step (b); and
d) identifying a drug candidate that performs said binding action of step (b) and said inhibition action of step (c).

2. The method according to claim 1 wherein at least one binding site of IKK-β is mutated; said mutated binding site is selected from a group consisting of phenylalanine residue, serine-177/181 residue, allosteric binding site of IKK-β, and cysteine residue except cysteine-46 residue.

3. The method according to claim 2 wherein said mutated cysteine or phenylalanine residue is selected from a group consisting of cysteine-12 residue, phenylalanine-26 residue, cysteine-59 residue, cysteine-99 residue, cysteine-114 residue, cysteine-115 residue, cysteine-179 residue, cysteine-215 residue, cysteine-299 residue, cysteine-370 residue, cysteine-412 residue, cysteine-444 residue, cysteine-464 residue, cysteine-524 residue, cysteine-618 residue, cysteine-662/716 residue, and cysteine-751 residue; said mutation is a point mutation from cysteine or phenylalanine to alanine.

4. The method according to claim 1 wherein said cancer is selected from a group consisting of lung cancer, colon cancer, liver cancer, breast cancer, prostate cancer, cervical cancer, acute promyelocytic leukemia (APL), acute myeloid leukemia (AML), acute lymphocytic leukemia (ALL), chronic myelogenous leukemia (CML), non-Hodgkin's lymphoma, Hodgkin's disease, chronic lymphocytic leukemia (CLL), myelodysplastic syndrome, Adult T-cell leukemia (ATL), Burkitt's lymphoma, B-cell lymphoma, primary malignant lymphocytes, B-cell chronic lymphocytic leukemia (B-CLL), human THP-1 leukemia, and multiple myeloma.

5. The method according to claim 1 wherein said inflammation is selected from a group consisting of ear edema, dermatitis, ear inflammation, and arthritis.

6. The method according to claim 1 wherein said neurodegenerative disease is selected from a group consisting of Alzheimer's disease, Parkinson's disease, amyotrophic lateral sclerosis, ataxia telangiectasia, spinocerebellar atrophy, multiple sclerosis, and Huntington's chorea.

7. The method according to claim 1 wherein said immunological disorder is selected from a group consisting of allergic rhinitis, allergic dermatitis, allergic contact dermatitis, allergic shock, asthma, papular urticaria, leucoderma, hypersensitivity vasculitis, hypersensitivity pneumonia, ulcerative colitis, glomerulonephritis, drug rashes, systemic lupus erythematosus, rheumatoid arthritis, scleroderma, multiple sclerosis, hyperthyroidism, idiopathic thrombocytopenic, autoimmune hemolytic anemia, allograft rejection, and hemolytic transfusion reaction.

8. The method according to claim 1 wherein said arthritic disorder is selected from a group consisting of rheumatoid arthritis, ankylosing spondylitis, gout, periarthritis, osteoarthritis, Reiter syndrome, psoriatic arthritis, post-traumatic arthritis, and enteropathic arthritis.

9. A method for diagnosing cancer, inflammation, neurodegenerative disease, immunological disorder, or arthritic disorder in a patient comprising:
a) obtaining a sample from said patient;
b) contacting said sample with a compound that binds to cysteine-46 residue of IKK-β of said sample;
c) detecting binding of said compound to IKK-β in said sample;
d) detecting inhibition action on kinase activity of IKK-β by said compound upon said binding in step (c); and
e) diagnosing said patient as having a likelihood to develop cancer, inflammation, neurodegenerative disease, immunological disorder, or arthritic disorder if said compound cannot perform said binding action of step (c) and/or said inhibition action of step (d).

10. The method according to claim 9 wherein at least one binding site of IKK-β is mutated; said mutated binding site is selected from a group consisting of phenylalanine residue, serine-177/181 residue, allosteric binding site of IKK-β, and cysteine residue except cysteine-46 residue.

11. The method according to claim 10 wherein said mutated cysteine or phenylalanine residue is selected from a group consisting of cysteine-12 residue, phenylalanine-26 residue, cysteine-59 residue, cysteine-99 residue, cysteine-114 residue, cysteine-115 residue, cysteine-179 residue, cysteine-215 residue, cysteine-299 residue, cysteine-370 residue, cysteine-412 residue, cysteine-444 residue, cysteine-464 residue, cysteine-524 residue, cysteine-618 residue, cysteine-662/716 residue, and cysteine-751 residue; said mutation is a point mutation from cysteine or phenylalanine to alanine.

12. The method according to claim 9 wherein said cancer is selected from a group consisting of lung cancer, colon cancer, liver cancer, breast cancer, prostate cancer, cervical cancer, acute promyelocytic leukemia (APL), acute myeloid leukemia (AML), acute lymphocytic leukemia (ALL), chronic myelogenous leukemia (CML), non-Hodgkin's lymphoma, Hodgkin's disease, chronic lymphocytic leukemia (CLL), myelodysplastic syndrome, Adult T-cell leukemia (ATL), Burkitt's lymphoma, B-cell lymphoma, primary malignant lymphocytes, B-cell chronic lymphocytic leukemia (B-CLL), human THP-1 leukemia, and multiple myeloma.

13. The method according to claim 9 wherein said inflammation is selected from a group consisting of ear edema, dermatitis, ear inflammation, arthritis.

14. The method according to claim 9 wherein said neurodegenerative disease is selected from a group consisting of Alzheimer's disease, Parkinson's disease, amyotrophic lateral sclerosis, ataxia telangiectasia, spinocerebellar atrophy, multiple sclerosis, and Huntington's chorea.

15. The method according to claim 9 wherein said immunological disorder is selected from a group consisting of allergic rhinitis, allergic dermatitis, allergic contact dermatitis, allergic shock, asthma, papular urticaria, leucoderma, hypersensitivity vasculitis, hypersensitivity pneumonia, ulcerative colitis, glomerulonephritis, drug rashes, systemic lupus erythematosus, rheumatoid arthritis, scleroderma, multiple sclerosis, hyperthyroidism, idiopathic thrombocytopenic, autoimmune hemolytic anemia, allograft rejection, and hemolytic transfusion reaction.

16. The method according to claim 9 wherein said arthritic disorder is selected from a group consisting of rheumatoid arthritis, ankylosing spondylitis, gout, periarthritis, osteoarthritis, Reiter syndrome, psoriatic arthritis, post-traumatic arthritis, and enteropathic arthritis.

17. A method of screening a patient to have a likelihood to develop cancer, inflammation, neurodegenerative disease, immunological disorder, or arthritic disorder comprising:
a) obtaining a sample from said patient;
b) contacting said sample with a compound that binds to cysteine-46 residue of IKK-β of said sample;
c) detecting binding of said compound to IKK-β in said sample;
d) detecting inhibition action on kinase activity of IKK-β by said compound upon said binding in step (d); and
e) identifying said patient as having a likelihood to develop cancer, inflammation, neurodegenerative disease, immunological disorder, or arthritic disorder if said compound cannot perform said binding action of step (c) and/or said inhibition action of step (d).

18. The method according to claim 17 wherein at least one binding site of IKK-β is mutated; said mutated binding site is selected from a group consisting of phenylalanine residue, serine-177/181 residue, allosteric binding site of IKK-β, and cysteine residue except cysteine-46 residue.

19. The method according to claim 18 wherein said mutated cysteine or phenylalanineresidue is selected from a group consisting of cysteine-12 residue, phenylalanine-26 residue, cysteine-59 residue, cysteine-99 residue, cysteine-114 residue, cysteine-115 residue, cysteine-179 residue, cysteine-215 residue, cysteine-299 residue, cysteine-370 residue, cysteine-412 residue, cysteine-444 residue, cysteine-464 residue, cysteine-524 residue, cysteine-618, cysteine-662/716 residue, and cysteine-751 residue; said mutation is a point mutation from cysteine or phenylalanine to alanine.

20. The method according to claim 17 wherein said cancer is selected from a group consisting of lung cancer, colon cancer, liver cancer, breast cancer, prostate cancer, cervical cancer, acute promyelocytic leukemia (APL), acute myeloid leukemia (AML), acute lymphocytic leukemia (ALL), chronic myelogenous leukemia (CML), non-Hodgkin's lymphoma, Hodgkin's disease, chronic lymphocytic leukemia (CLL), myelodysplastic syndrome, Adult T-cell leukemia (ATL), Burkitt's lymphoma, B-cell lymphoma, primary malignant lymphocytes, B-cell chronic lymphocytic leukemia (B-CLL), human THP-1 leukemia, and multiple myeloma.

21. The method according to claim 17 wherein said inflammation is selected from a group consisting of ear edema, dermatitis, ear inflammation, arthritis.

22. The method according to claim 17 wherein said neurodegenerative disease is selected from a group consisting of Alzheimer's disease, Parkinson's disease, amyotrophic lateral sclerosis, ataxia telangiectasia, spinocerebellar atrophy, multiple sclerosis, and Huntington's chorea.

23. The method according to claim 17 wherein said immunological disorder is selected from a group consisting of allergic rhinitis, allergic dermatitis, allergic contact dermatitis, allergic shock, asthma, papular urticaria, leucoderma, hypersensitivity vasculitis, hypersensitivity pneumonia, ulcerative colitis, glomerulonephritis, drug rashes, systemic lupus erythematosus, rheumatoid arthritis, scleroderma, multiple sclerosis, hyperthyroidism, idiopathic thrombocytopenic, autoimmune hemolytic anemia, allograft rejection, and hemolytic transfusion reaction.

24. The method according to claim 17 wherein said arthritic disorder is selected from a group consisting of rheumatoid arthritis, ankylosing spondylitis, gout, periarthritis, osteoarthritis, Reiter syndrome, psoriatic arthritis, post-traumatic arthritis, and enteropathic arthritis.

25. A method for treating cancer, inflammation, neurodegenerative disease, immunological disorder, or arthritic disorder comprising administering an effective amount of a therapeutic agent to a patient in need thereof, wherein said patient harbors gene mutations on at least one binding site of IKK-β; said mutated binding site is selected from a group consisting of phenylalanine residue, serine-177/181 residue, allosteric binding site of IKK-β, and cysteine residue except cysteine-46 residue.

26. The method according to claim 25 wherein said mutated cysteine or phenylalanineresidue is selected from a group consisting of cysteine-12 residue, phenylalanine-26 residue, cysteine-59 residue, cysteine-99 residue, cysteine-114 residue, cysteine-115 residue, cysteine-179 residue, cysteine-215 residue, cysteine-299 residue, cysteine-370 residue, cysteine-412 residue, cysteine-444 residue, cysteine-464 residue, cysteine-524 residue, cysteine-618 residue, cysteine-662/716 residue, and cysteine-751 residue; said mutation is a point mutation from cysteine or phenylalanine to alanine.

27. The method according to claim 25 wherein said therapeutic agent binds to cysteine-46 residue of IKK-β and inhibits the kinase activity of IKK-β upon said binding.

28. The method according to claim 25 wherein said cancer is selected from a group consisting of lung cancer, colon cancer, liver cancer, breast cancer, prostate cancer, cervical cancer, acute promyelocytic leukemia (APL), acute myeloid leukemia (AML), acute lymphocytic leukemia (ALL), chronic myelogenous leukemia (CML), non-Hodgkin's lymphoma, Hodgkin's disease, chronic lymphocytic leukemia (CLL), myelodysplastic syndrome, Adult T-cell leukemia (ATL), Burkitt's lymphoma, B-cell lymphoma, primary malignant lymphocytes, B-cell chronic lymphocytic leukemia (B-CLL), human THP-1 leukemia, and multiple myeloma.

29. The method according to claim 25 wherein said inflammation is selected from a group consisting of ear edema, dermatitis, ear inflammation, arthritis.

30. The method according to claim 25 wherein said neurodegenerative disease is selected from a group consisting of Alzheimer's disease, Parkinson's disease, amyotrophic lateral sclerosis, ataxia telangiectasia, spinocerebellar atrophy, multiple sclerosis, and Huntington's chorea.

31. The method according to claim 25 wherein said immunological disorder is selected from a group consisting of allergic rhinitis, allergic dermatitis, allergic contact dermatitis, allergic shock, asthma, papular urticaria, leucoderma, hypersensitivity vasculitis, hypersensitivity pneumonia, ulcerative colitis, glomerulonephritis, drug rashes, systemic lupus erythematosus, rheumatoid arthritis, scleroderma, multiple sclerosis, hyperthyroidism, idiopathic thrombocytopenic, autoimmune hemolytic anemia, allograft rejection, and hemolytic transfusion reaction.

32. The method according to claim 25 wherein said arthritic disorder is selected from a group consisting of rheumatoid arthritis, ankylosing spondylitis, gout, periarthritis, osteoarthritis, Reiter syndrome, psoriatic arthritis, post-traumatic arthritis, and enteropathic arthritis.
